(19) United States
(12) Patent Application Publication
(10) Pub. No.: US 2018/0256494 A1
(43) Pub. Date: Sep. 13, 2018

(54) TOPICAL COMPOSITION FOR TREATING AN INFLAMMATORY SKIN DISEASE AND A METHOD FOR TREATING THE INFLAMMATORY SKIN DISEASE WITH THE SAME

(71) Applicants: Beyong Deog PARK, Bethlehem, PA (US); Se Kyoo JEONG, Daejeon (KR); Kyung Ho PARK, Seoul (KR); Kyong Oh SHIN, ChungBuk (KR); Hyun Jong KIM, Englewood Cliffs, NJ (US)

(72) Inventors: Beyong Deog PARK, Bethlehem, PA (US); Se Kyoo JEONG, Daejeon (KR); Kyung Ho PARK, Seoul (KR); Kyong Oh SHIN, ChungBuk (KR); Hyun Jong KIM, Englewood Cliffs, NJ (US)

(21) Appl. No.: 15/917,496

(22) Filed: Mar. 9, 2018

Related U.S. Application Data
(60) Provisional application No. 62/469,762, filed on Mar. 10, 2017.

(51) Int. Cl. A61K 9/00; A61P 17/00; A61P 29/00; A61K 31/155; A61K 31/436; A61K 31/573; A61K 31/59

(52) U.S. Cl. CPC A61K 9/0014; A61P 17/00; A61P 29/00; A61K 31/59; A61K 31/436; A61K 31/573; A61K 31/155

(57) ABSTRACT

A topical composition for treating an inflammatory skin disease in a subject includes a therapeutically effective amount of a biguanidine, and is in a dosage form for topical administration. A method for treating an inflammatory skin disease in a subject includes the following steps: identifying a subject in need thereof; providing a biguanidine; and administering the biguanidine in a therapeutically effective amount to a part of the subject to treat the inflammatory skin disease.

The present application claims priority to U.S. Provisional Patent Application No. 62/469,762, filed on Mar. 10, 2017, which is incorporated by reference for all purposes as if fully set forth herein.

### BACKGROUND OF THE INVENTION

### Field of the Invention

The present invention relates to a topical composition for treating an inflammatory skin disease and a method for treating the inflammatory skin disease with the same.

### Discussion of the Related Art

Inflammatory skin diseases include, for example, eczema, psoriasis, rosacea, acne vulgaris, ulcers, seborrhoeic dermatitis, and irritations. Methotrexate is used to control severe psoriasis or rheumatoid arthritis that has not responded to other treatments. It belongs to a class of drugs known as antimetabolites.

Common adverse effects of methotrexate include liver damage, ulcerative stomatitis, low white blood cell count, nausea, abdominal pain, fatigue, fever, dizziness, acute pneumonitis, rarely pulmonary fibrosis and kidney failure. Methotrexate is harmful to fetus and not used in pregnancy. Methotrexate's adverse effects also include myelopathies, leucoencephalopathies, neurological damage and memory loss.

There are needs to develop new and effective agents to treat various inflammatory skin diseases. Specially, these agents need have low adverse effects.

### SUMMARY OF THE INVENTION

An embodiment of the present invention is a topical composition for treating an inflammatory skin disease in a subject. The topical composition includes a therapeutically effective amount of a biguanidine, and is in a dosage form for topical administration. The biguanidine can be biguanide or metformin. The topical composition further includes a topical calcineurin inhibitor (TCIs), such as tacrolimus and pimecrolimus, a topical glucocorticoid, such as hydrocortisone, mometasone fuorate, dexamethasone, and clobetasol 17-propionate, or a topical vitamin D analogue, such as calcipotriol. The topical biguanidine activates autophagy, which results in alleviation of local inflammation. The biguanidine can be in an amount between 0.001% and 20%, 0.005% and 10%, or 0.01% and 5%, by weight of the total topical composition.

Another embodiment of the present invention is a method for treating an inflammatory skin disease in a subject. The method includes the steps of: identifying a subject in need thereof; providing a topical composition that includes a therapeutically effective amount of a biguanidine; and administering the topical composition to a part of the subject to treat the inflammatory skin disease. The biguanidine can be a biguanide or metformin. The method further includes providing and administering a compound of a topical calcineurin inhibitor (TCIs), such as tacrolimus and pimecrolimus, a topical glucocorticoid, such as hydrocortisone, mometasone fuorate, dexamethasone, and clobetasol 17-propionate, or a topical vitamin D analogue, such as calcipotriol. The topical biguanidine activates autophagy, which results in alleviation of local inflammation. The biguanidine is in an amount between 0.001% and 20%, 0.005% and 10%, or 0.01% and 5% by weight of the total topical composition.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 shows the Trans-Epidermal Water Loss (TEWL) value changes in oxazolone-induced chronic dermatitis animal model.

FIG. 2 shows the skin thickness measurement data in oxazolone-induced chronic dermatitis animal model.

### DETAILED DESCRIPTION OF THE ILLUSTRATED EMBODIMENTS

Reference will now be made in detail to embodiments of the present invention, example of which is illustrated in the accompanying drawings.

The inventors of the present invention discovered that the topical composition of biguanidine can treat inflammatory skin diseases. Without being bound by theory, it is hypothesized that autophagy plays important roles in inflammation and the activation of autophagy can alleviate inflammatory responses, and biguanidine can activate autophagy and reduce inflammation.

Autophagy (“self-eating” in Greek) is self-digestive process that targets internal or damaged organelles and misfolded protein to lysosomal degradation. Autophagy has housekeeping function to remove aggregated proteins and damaged organelles. In response to cellular stress, such as metabolic stress or nutrition deprivation, autophagy supplies cells with energy and essential metabolic materials to maintain normal cellular functions.

The autophagy pathways and proteins play a role in the control of inflammatory signaling. Autophagy contributes to host defense responses by promoting the elimination of pathogens and the induction of acquired immunity. Another effect of autophagy proteins on inflammatory signaling relates to the regulation of the inflammasome-dependent responses. Autophagy regulates these responses by controlling the levels of pro-inflammatory cytokine expression. Active autophagy reduces inflammation at least by mediating damaged organelles clearance, lowering micro-organelles intracellular load and degrading inflammatory mediators.

5′-AMP-activated protein kinase (AMPK) has a key role in the regulation of cellular lipid and protein metabolism in response to stimuli such as exercise and changes in fuel availability. AMPK acts as an energy sensor in cellular metabolism, and inhibits energy-consuming functions. It plays in important role in the maintenance of cellular energy homeostasis and the activation of autophagy. The role of the AMPK is mainly to control metabolism based on nutrient supply. AMPK is involved in the regulation of inflammatory responses and has controlling activity in dendritic cells, T-lymphocytes, macrophages, endothelial cells and monocytes.

Biguanidine is a class of compounds that function as oral antihyperglycemic drugs used for diabetes mellitus or predidabetes treatment. Biguanidine includes, for example, biguanide, metformin, phenformin, and buformin. Metformin is a first-line treatment in the management of insulin resistance and type 2 diabetes mellitus.

Biguanidine activates AMPK. AMPK consists of three subunits: α, β, and γ subunits. The γ subunit is functioning as a cellular AMP or ADP sensor, which changes its conformation upon binding AMP or ZMP or other activators such as salicylate and increases the availability of the α subunit to be substrate of upstream protein kinases. Biguanidine binds to the γ subunit of AMPK in vitro. Biguanidine does not activate AMPK in cells where the γ subunit is manipulated to be unresponsive to AMP. Thus, AMPK is activated by lowering the cellular energy status and changing the ATP /AMP ratio via mitochondrial inhibition.

Biguanidine can activate autophagy and reduce inflammation, and thus can treat inflammatory skin diseases. Biguanidine also has some side effects. For example, adverse reactions are frequent in patients taking metformin via oral administration route. Notable adverse reactions include gastrointestinal side effects such as anorexia, nausea, vomiting, abdominal discomfort and diarrhea. For example, many patients taking metformin report a variety of unpleasant side effects: nausea, diarrhea, bloating, gastrointestinal distress. Increased liver enzymes are also some side effects that occur in patients. Because of the side effects, many patients are either not as optimally complaint with therapy as they could be, or simply stop taking the medication altogether.

The inventors discovered a new delivery method that allows a patient all the benefit of anti-inflammation effects of biguanidine, while reducing or eliminating the side effects associated with orally-administrated biguanidine. The new delivery method of biguanidine is topical composition. An advantage of the topical composition is that the dose topical composition can be 5%, 10%, 15%, 20%, 25%, 30%, 35%, 40%, 45%, 50%, 55%, 60%, 65%, 70%, 75% or 80% of the oral dose. Doctors and patients can adjust the doses based on the severity of the skin inflammation. Because of the low dose of the topical composition, the side effects can be avoided or eliminated.

A topical composition for treating an inflammatory skin disease in a patient includes a therapeutically effective amount of a biguanidine, and is in a dosage form for topical administration. The term “therapeutically effective amount” refers to that amount which is sufficient to effect treatment, as defined above, when administered to a subject (e.g. a mammal) in need of such treatment. The therapeutically effective amount will vary depending on the subject and disease state being treated, the severity of the affliction and the manner of administration, and may be determined routinely by one of ordinary skill in the art. Thus, a therapeutically amount of a biguanidine is that amount which is sufficient to effect treatment, as defined above, when administered to a subject in need of treatment by a biguanidine.

A topical composition is a composition that is applied to a particular place on or in the body, for example, application to body surfaces such as the skin or mucous membranes. Topical compositions include a large range of classes, for examples, creams, foams, gels, lotions, and ointments. Some topical compositions are epicutaneous, i.e., being applied directly to the skin. Some topical compositions are inhalational (e.g., asthma treatment compositions) which are applied to the surface of tissues other than the skin. Topical compositions may have a local target or have systemic effects.

The topical composition of biguanidine is designed to provide anti-inflammation effects in the affected tissues with dissemination throughout the affected area to be treated, with little to no increase in systemic blood levels of the biguanidine. The topical composition can consist solely of the biguanidine, or the biguanidine combined with one or more excipients, preferably for topical transdermal administration. The formulation can also further include other constituents such as penetration enhancers or other active ingredients. The preferred topical composition contains the biguanidine in the form of micro or nanoparticles, which may be formed of drug alone or in combination with an excipient or carrier. The biguanidine formulation may be in the administered as a cream, lotion, ointment, emulsion, shea butter, gel, suspension, solution or transdermal patch.

The biguanidine refers to a class of compounds that function as oral antihyperglycemic drugs used for diabetes mellitus or prediabetes treatment. Examples of the biguanidine include biguanide, metformin, phenformin, and buformin. For the topical composition, the biguanidine can be biguanide or metformin. Biguanide is the organic compound with the formula

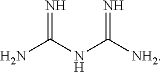

Metformin is the organic compound with the formula

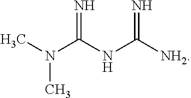

The topical composition further includes topical calcineurin inhibitors (TCIs) such as tacrolimus, pimecrolimus, topical glucocorticoids such as hydrocortisone, mometasone fuorate, dexamethasone, clobetasol 17-propionate, or topical vitamin D analogues such as calcipotriol. The biguanidine can be in an amount between 0.001% and 20%, 0.005% and 10%, or 0.01% and 5%, by weight of the total topical composition.

A method for treating an inflammatory skin disease in a patient includes the steps of: identifying a patient in need thereof; providing a topical composition that includes a therapeutically effective amount of a biguanidine; and administering the topical composition to a part of the patient to treat the inflammatory skin disease.

### REPRESENTATIVE EXAMPLES

### Example 1

### Trans-Epidermal Water Loss (TEWL) Measurement in Oxazolone-Induced Chronic Dermatitis Animal

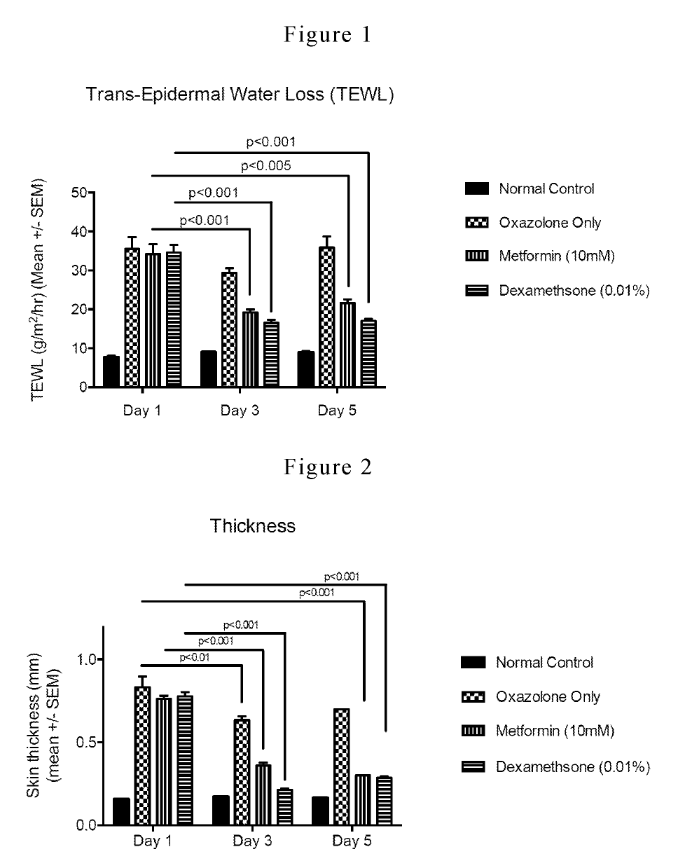

The test was conducted with Normal Control, Oxazolone Only treated group, Metformin (topical, 10 mM), and Dexamethsone (0.01%), and the results are shown in FIG. 1. The test measures the quantity of water that passes from inside a body through the epidermal layer (skin) to the surrounding atmosphere via diffusion and evaporation processes. Oxazolone (OXA), a hapten that can induce allergic contact dermatitis-like or atopic dermatitis-like skin inflammation, was used to elucidate the anti-inflammatory action of Metformin (10 mM) and Dexamethsone (0.01%).

The results show that the topical application of Metformin (10 mM) has similar effect as that of topical Dexamethsone (0.01%) at Day 1, Day 3, and Day 5. It is noted that Dexamethsone (0.01%) has adverse effects also include myelopathies, leucoencephalopathies, neurological damage and memory loss. Metformin (10 mM) has significantly lower adverse effects than Dexamethsone (0.01%).

### Example 2

### Thickness Test in OXA

The test was conducted with Normal Control, Oxazolone-Only treated group, Metformin (topical, 10 mM), and Dexamethsone (0.01%), and the results are shown in FIG. 2. Ear swelling, or hyperplasia, is a feature of a topic dermatitis. Epidermis thickening can be one of the factors that contribute to the ear swelling. In the OXA-mediated dermatitis model, the thickening of epidermis was observed.

Again, the results show that topical Metformin (10 mM) has similar effect as that of topical Dexamethsone (0.01%) at Day 1, Day 3, and Day 5. It is noted that Dexamethsone (0.01%) has adverse effects also include myelopathies, leucoencephalopathies, neurological damage and memory loss. Metformin (10 mM) has significantly lower adverse effects than Dexamethsone (0.01%).

Examples 1 and 2 show that Metformin (10 mM) is effective to treat skin inflammation and has low adverse effects.

### Example 3

### Combination of Metformin and a Topical Calcineurin Inhibitor

The tests can be conducted in the same way as Examples 1 and 2, except that Metformin (10 mM) is replaced with a combination of Metformin and a topical calcineurin inhibitor. The topical calcineurin inhibitor can be tacrolimus.

The synergistic effect of adding Metformin along with tacrolimus can be observed.

### Example 4

### Combination of Metformin and a Topical Calcineurin Inhibitor

The tests can be conducted in the same way as Examples 1 and 2, except that Metformin (10 mM) is replaced with a combination of Metformin and a topical calcineurin inhibitor. The topical calcineurin inhibitor can be pimecrolimus.

The synergistic effect of adding Metformin along with pimecrolimus can be observed.

### Example 5

### Combination of Metformin and a Topical Glucocorticoid

The tests can be conducted in the same way as Examples 1 and 2, except that Metformin (10 mM) is replaced with a combination of Metformin and a topical glucocorticoid. The topical glucocorticoid can be hydrocortisone.

The synergistic effect of adding Metformin along with hydrocortisone can be observed.

### Example 6

### Combination of Metformin and a Topical Glucocorticoid

The tests can be conducted in the same way as Examples 1 and 2, except that Metformin (10 mM) is replaced with a combination of Metformin and a topical glucocorticoid. The topical glucocorticoid can be mometasone fuorate.

The synergistic effect of adding Metformin along with mometasone fuorate can be observed.

### Example 7

### Combination of Metformin and a Topical Glucocorticoid

The tests can be conducted in the same way as Examples 1 and 2, except that Metformin (10 mM) is replaced with a combination of Metformin and a topical glucocorticoid. The topical glucocorticoid can be dexamethasone.

The synergistic effect of adding Metformin along with dexamethasone can be observed.

### Example 8

### Combination of Metformin and a Topical Glucocorticoid

The tests can be conducted in the same way as Examples 1 and 2, except that Metformin (10 mM) is replaced with a combination of Metformin and a topical glucocorticoid. The topical glucocorticoid can be clobetasol 17-propionate.

The synergistic effect of adding Metformin along with clobetasol 17-propionate can be observed.

### Example 8

### Combination of Metformin and a Topical Vitamin D Analogue

The tests can be conducted in the same way as Examples 1 and 2, except that Metformin (10 mM) is replaced with a combination of Metformin and a topical vitamin D analogue. The topical vitamin D analogue can be calcipotriol.

The synergistic effect of adding Metformin along with calcipotriol can be observed.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. A topical composition for treating an inflammatory skin disease in a subject, the topical composition comprising a therapeutically effective amount of a biguanidine, wherein the composition is in a dosage form for topical administration.

2. The topical composition of claim 1, wherein the biguanidine is biguanide or metformin.

3. The topical composition of claim 1, further comprising a compound selected from the group consisting of a topical calcineurin inhibitor, a topical glucocorticoid, and a topical vitamin D analogue.

4. The topical composition of claim 3, wherein
the topical calcineurin inhibitor is tacrolimus or pimecrolimus,
the topical glucocorticoid is hydrocortisone, mometasone fuorate, dexamethasone, or clobetasol 17-propionate, and
the topical vitamin D analogue is calcipotriol.

5. The topical composition of claim 1, wherein the biguanidine activates autophagy and reduces inflammation.

6. The topical composition of claim 1, wherein the biguanidine is in an amount between 0.001% and 20% by weight of the total topical composition.

7. The topical composition of claim 6, wherein the biguanidine is in an amount between 0.005% and 10% by weight of the total topical composition.

8. The topical composition of claim 7, wherein the biguanidine is in an amount between 0.01% and 5% by weight of the total topical composition.

9. A method for treating an inflammatory skin disease in a subject, comprising the steps of:
identifying a subject in need thereof;
providing a topical composition, the topical composition including a therapeutically effective amount of a biguanidine; and
administering the topical composition to a part of the subject to treat the inflammatory skin disease.

10. The method of claim 9, wherein the biguanidine is biguanide or metformin.

11. The method of claim 9, further comprising providing and administering a compound selected from the group consisting of a topical calcineurin inhibitor, a topical glucocorticoid, and a topical vitamin D analogue.

12. The method of claim 11, wherein
the topical calcineurin inhibitor is tacrolimus or pimecrolimus,
the topical glucocorticoid is hydrocortisone, mometasone fuorate, dexamethasone, or clobetasol 17-propionate, and
the topical vitamin D analogue is calcipotriol.

13. The method of claim 9, wherein the biguanidine activates autophagy and reduces inflammation.

14. The method of claim 9, wherein the biguanidine is in an amount between 0.001% and 20% by weight of the total topical composition.

15. The method of claim 14, wherein the biguanidine is in an amount between 0.005% and 10% by weight of the total topical composition.

16. The method of claim 15, wherein the biguanidine is in an amount between 0.01% and 5% by weight of the total topical composition.
